(19) United States
(12) Patent Application Publication
(10) Pub. No.: US 2007/0094050 A1
(43) Pub. Date: Apr. 26, 2007

(54) Method for linking sets of data comprising medical therapeutic indications

(76) Inventors: Klaus Abraham-Fuchs, Erlangen (DE); Eva Rumpel, Erlangen (DE)

Correspondence Address: HARNESS, DICKEY & PIERCE, P.L.C., P.O.BOX 8910, RESTON, VA, 20195, US

(21) Appl. No.: 10/557,473

(22) PCT Filed: May 6, 2004
(86) PCT No.: PCT/EP04/04846

§ 371 Date: Nov. 24, 2006

(30) Foreign Application Priority Data
May 20, 2003 (DE) 103 22 686.9

(51) Int. Cl. G06F 19/00; G09B 23/28; A61B 5/00; G06F 7/00

(52) U.S. Cl. 705/3; 600/300; 434/262; 707/101

(57) ABSTRACT

A method is for linking sets of data including medical therapeutic indications. According to the method, the respective set of data of a therapeutic indication is provided with at least one output while a reference regarding a therapeutic indication that follows in a treatment process is allocated to an output such that any link can be created between therapeutic indications which succeed each other in a meaningful manner in the treatment process.

This application is the national phase under 35 U.S.C. § 371 of PCT International Application No. PCT/EP2004/004846 which has an International filing date of May 6, 2004, which designated the United States of America and which claims priority on European Patent Application number EP 103 22 686.9 filed May 20, 2003, the entire contents of which are hereby incorporated herein by reference.

### FIELD

The invention generally relates to a method for linking sets of data or data records including medical therapeutic advice items or indications. Such data records are themselves called a therapeutic advice item or indication below, for short. The data records may include both data, that is to say therapeutic advice items or indications, and executable code, that is to say software fragments, e.g. in the form of expert rules.

### BACKGROUND

In recent years, “medical guidelines” (known as medical practice guidelines or clinical practice guidelines), guidelines for short, have become increasingly significant. Guidelines are diagnostic and/or therapeutic recommendations for action and decisions (referred to generally and in summary below as therapeutic advice items) to the doctor which are drawn up in a broad consensus by superordinate, generally recognized, committees in the medical profession.

Naturally, such therapeutic advice items need to be constantly brought into line with the latest level of knowledge, and are thus subject to constant, if usually long-term or medium-term, change. Typically, therapeutic advice items are revised and brought into line at intervals of a few years. A medical practitioner, that is to say a doctor, therapist or the like, who treats patients by making diagnostic and therapeutic decisions on the basis of such established diagnosis or therapeutic advice items is now confronted by the challenge of obtaining an overview of which therapeutic advice items are related such that successive application results in appropriate and promising treatment.

For a detailed explanation of therapeutic advice items and their dependencies and interactions, reference is made to the patent application entitled “Method for referencing data records which comprise therapeutic advice items” submitted on the same date by the same applicant.

To solve these problems, no automatically executable methods have been disclosed in the prior art to date to the best of the applicant's knowledge. In addition, widespread use of therapeutic advice items, particularly in the form of guidelines, is in its infancy in medicine.

### SUMMARY

At least one embodiment of the invention relates to an improvement in the opportunities for using therapeutic advice items by demonstrating a way of linking therapeutic advice items and hence simulating an ordinary process when treating patients.

At least one embodiment of the invention may achieve an object via a method. In this regard, a method of at least one embodiment is for linking data records and includes medical therapeutic advice items provides for the respective data record associated with a therapeutic advice item to include at least one output and for an output to be assigned a reference to a data record for a subsequent therapeutic advice item in a treatment process.

In its most general form, a data record for a therapeutic advice item may include a collection of data—such as input and/or output variables for therapeutic information and a number of expert rules—which, on the basis of therapeutic information, e.g. using patient data and/or using data about available diagnostic and/or therapeutic device, are used to generate a therapeutic advice item using the expert rules. The data may be provided for the purpose of generating a therapeutic advice item in the form of input data or user data. The data record may bed provided with a reference which is used to access a subsequent data record in order to generate a further subsequent therapeutic advice item or which is used to access subsequent data, such as therapeutic information, e.g. further required patient data and/or required expert rules, in order to add to the therapeutic advice item in question.

Generation of the therapeutic advice item therefore results in activation of the appropriate output of the data record for the therapeutic advice item. Depending on the respective therapeutic advice item, that is to say the respectively activated output, different options for continuing the therapy may arise. For this reason, at least one output of the data record for the therapeutic advice item in question is assigned a reference to a data record for a subsequent therapeutic advice item in the treatment process. When this output is activated, the referenced therapeutic advice item can be selected.

At least one embodiment of the invention includes insight that the use of modern information and communication technology is currently being greatly expanded in the health service in parallel and independently of the introduction of guidelines in medicine. The use of electronic data processing in hospitals (e.g. HIS=Hospital Information System, RIS—Radiology Information System, PACS=Picture Archive & Communication System, LIS=Laboratory Information System) and in doctor's practices (practice management software, electronic patient records) is becoming more and more usual. A subsequent development step is generally expected to network this software and these databases across the institutions in the health service (clinics, doctor's practices, therapeutic practices etc.). This provides the option of a “networked health service”, at first at national or regional level and later globally. This development provides the basis for the use of the two aspects of embodiments of the invention.

At least one advantage of at least one embodiment of the invention and its refinements is that full treatment processes with individual treatment sequences can be depicted in the form of linked therapeutic advice items. This makes the use of therapeutic advice items even more attractive to medical practitioners, that is to say doctors, therapists etc.

A particularly advantageous form of at least one embodiment of the referencing of data records for therapeutic advice items can occur, for example, if each data record for a therapeutic advice item is assigned an order feature provided for identifying the latter, and the referencing relates to the order feature of the data record for the therapeutic advice item which is to be referenced.

An alternative, likewise advantageous, form of the referencing of data records for therapeutic advice items is possible if each data record for a therapeutic advice item includes at least one input (also called input variable) having a unique input identifier, and the referencing relates to an input identifier for the data record for the therapeutic advice item which is to be referenced.

Preferably, a data record for a therapeutic advice item has a plurality of outputs, with at least one output being activated, particularly on the basis of the underlying therapeutic information, such as patient data and/or diagnostic and/or therapeutic device, so that defined dependencies are produced between the referencing and the referenced therapeutic advice item. In this context, a single output of a data record for a therapeutic advice item can reference a plurality of data records for different therapeutic advice items, however.

If there is not already a link between data records for a plurality of therapeutic advice items, it is necessary to make such links for the individual users of the therapeutic advice items oneself. To avoid links made manually in this manner and hence to avoid possibly incorrect links, the data record for a therapeutic advice item is referenced by using the order feature of the data record for the referencing therapeutic advice item in order to select, automatically, further data records for referenceable therapeutic advice items which have partially identical order features and by proposing them to the user for selection, where the data record for the referencing therapeutic advice item is assigned the order feature of the relevant therapeutic advice item's data record selected in this manner. In this case, it is sufficient for just portions of the entire length of order features of corresponding therapeutic advice items to match. By way of example, it is sufficient for there just to be a match in a “diabetes” portion of the order features, e.g. 231-diabetes-I-V031-May 2000 or 234-diabetes-II-V027-July 2001, which is then used to make the selection and assignment of a further therapeutic advice item which likewise has this portion of the order features.

Alternatively, this can also be done equally efficiently if a data record for a therapeutic advice item is referenced by using an output identifier for the activated output of the data record for the referencing therapeutic advice item to select, automatically, referenceable therapeutic advice items which have an input identifier which is partially identical or similar to the output identifier of the activated output and by proposing them to the user for selection, and the data record for the referencing therapeutic advice item is assigned, at its activated output, the input identifier of the therapeutic advice item selected in this manner. In similar fashion to the selection and assignment and hence to the linking of therapeutic advice items using partially matching order features, it is also sufficient when using the input identifier for linking if at least a “diabet” portion of the input identifier, e.g. ICDXXX-diabetes, ICDYY-diabetic retinopathy, matches the therapeutic advice items in question.

The linked therapeutic advice items may, in at least one embodiment, be advantageously applied in the manner described below using a method for linking data records comprising medical therapeutic advice items, where a patient is treated by applying a first data record comprising therapeutic information and by prescribing at least one reference to a second data record for a therapeutic advice item of the type described above. In a first step, the second data record for the referenced therapeutic advice item processes selected patient data from the first data record. In a second step, the second data record associated with the therapeutic advice item uses the patient data from the first data record to activate at least one associated output, and if the output in question comprises a reference to a further second data record for a further therapeutic advice item then the process continues with the first step. The second data record for the referenced therapeutic advice item (that is to say now the second therapeutic advice item, which is subsequent to the first therapeutic advice item) in turn processes selected patient data and/or information about diagnostic and/or therapeutic means from the first data record and then activates an associated output.

If this output again also comprises a reference to a further second data record for a further therapeutic advice item, the method is continued with this next second data record for the referenced therapeutic advice item. The method is terminated when an output is activated which comprises no reference to a subsequent therapeutic advice item. Another particular feature is that in the first step, the expression “referenced therapeutic advice item” relates to the stipulation of referencing for a therapeutic advice item the first time that the first step is carried out, and relates to referencing associated with the respective preceding activated output each time the first step is carried out again.

Preferably, the therapeutic information, particularly the patient data, is stored in a prescribed format or the patient data are assigned a prescribed identifier, so that it is possible for the respective patient data required for applying a therapeutic advice item to be selected from the total available patient data automatically.

A particularly efficient way of automatically selecting the necessary patient data, in at least one embodiment, is provided if the second data record for a therapeutic advice item includes at least one input and a format associated with this input or an identifier associated with this input, and the format or the identifier for the or each input, on the one hand, and the format or the identifier for the patient data in the first data record, on the other hand, are used to select the patient data to be processed.

A further advantage of at least one embodiment of the invention and of its refinements is, in particular, that the medical practitioner making the links for the individual therapeutic advice items himself can use the automatically generated list containing referenceable therapeutic advice items to obtain suggestions for variations in the therapy.

### BRIEF DESCRIPTION OF THE DRAWINGS

An example embodiment of the invention is explained in more detail below with reference to the drawings. Mutually corresponding objects or elements are provided with the same reference symbols in all the figures, in which:

FIGS. 1 to 4 show a schematic illustration of various alternative embodiments for software-based linking of data records including therapeutic advice items.

Mutually corresponding parts are provided with the same reference symbols in all the figures.

### DETAILED DESCRIPTION OF THE EXAMPLE EMBODIMENTS

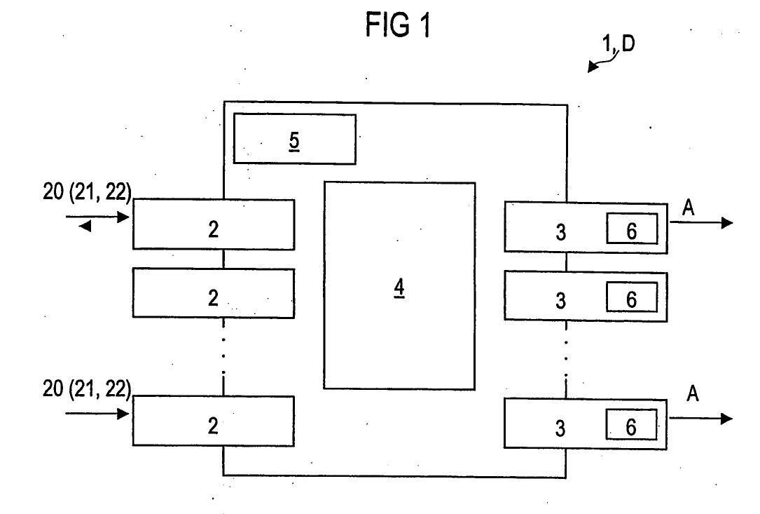

FIG. 1 shows a schematic illustration of the software-based implementation of the basic structure of a medical guideline, using a data record D for a therapeutic advice item 1. The data record D (subsequently called therapeutic advice item 1 merely for brevity) includes at least one input 2 (also called input variable) and at least one output 3. The or each input 2 is used for directly or indirectly supplying the therapeutic advice item 1 with therapeutic information 20, such as patient data 21 or information 22, about diagnostic and/or therapeutic device/method/etc.

The core of the therapeutic advice item 1 is formed by an expert rule 4. Using the expert rule 4, single or a plurality of outputs 3 are activated on the basis of the therapeutic information 20 at the or each input 2, and hence individual, i.e. patient-related or device-related, recommendations A for action and/or decisions are output in the form of procedures or instructions for action.

The therapeutic information 20 at an input 2, that is to say the patient data 21 or information 22, may be from various categories, e.g. measured data such as blood pressure, electrocardiogram, available technical device(s), such as X-ray unit, etc., insights such as a previous diagnosis or actions such as an implemented therapeutic option etc. Similarly, the individual recommendations A for action and/or decisions which are output at the outputs 3 may be from various categories, e.g. an insight such as a diagnosis derived from the input values for the therapeutic information 20, a recommendation for action or further data derived from input data for the therapeutic information 20.

The expert rule 4 is generally what is known as an inference rule, which is used to derive single or a plurality of individual—patient-related and/or device-related—recommendations A for action and/or decisions from the therapeutic information 20 (also called input data), the recommendations resulting in activation of the respective output 3 or of the respective outputs 3 and, if appropriate, being output at said output.

In the example embodiment, each therapeutic advice item 1 includes an order feature 5 uniquely identifying the therapeutic advice item 1. The order feature 5 is suitable and provided for referencing a therapeutic advice item 1 which is stored in a central database (not shown) and is used in a medical institution, for example.

Each output 3 has an associated reference 6 for referencing a further therapeutic advice item 1. If no further therapeutic advice item 1 is to be linked when an output 3 is activated, the value of the reference 6 is set to a prescribed value, e.g. zero. If a subsequent therapeutic advice item 1 is to be linked when an output 3 is activated, the reference 6 entered is the order feature 5 of the therapeutic advice item.

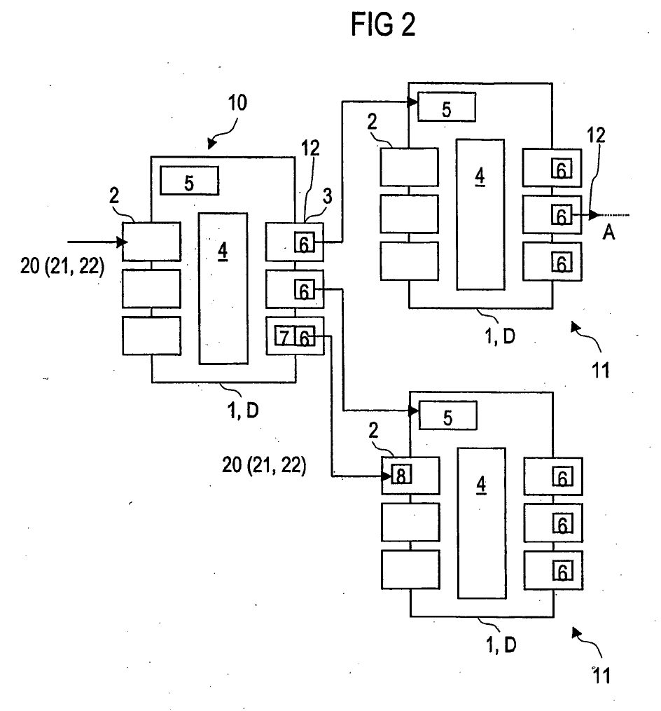

FIG. 2 shows an example of linked therapeutic advice items 1. A first therapeutic advice item 1 which is referenced to therapeutic information (not shown), particularly using the order feature 5, (in the illustration the therapeutic advice item 1 on the left) includes three outputs 3 by way of example. Two of these outputs 3 include a reference 6 which is used to reference a respective subsequent therapeutic advice item 1 (in the illustration the therapeutic advice items 1 on the right). Alternatively, it is also possible for the reference 6 entered in the form of an output identifier 7 to be an input identifier 8 (for example for therapeutic information 20) which is associated with a particular input 2 of the subsequent therapeutic advice item 1.

The therapeutic advice item 1 which references a subsequent therapeutic advice item 1 for linking purposes is called the referencing therapeutic advice item 10 in order to distinguish it. The therapeutic advice item 1 to which the referencing relates is accordingly called the referenced therapeutic advice item 11 in order to distinguish it. The output 3 of a therapeutic advice item 1 which is activated when the expert rule 4 underlying the therapeutic advice item 10 is applied is called the activated output 12 in order to distinguish it.

For referencing purposes, the reference 6 assigned to the respective output 12 is the order feature 5 of the referenced therapeutic advice item 11. This can be done most simply by virtue of the reference 6 entered being the order feature 5 of the referenced therapeutic advice item 11. This requires a “look-up table” which includes all the order features 5 of the available therapeutic advice items 1, 10, 11 and, for each order feature 5, a reference 6 to the respective therapeutic advice item 11, particularly in the form of the storage location, that is to say an address. Alternatively, provision may also be made for the reference 6 entered to be the storage location of the referenced therapeutic advice item 11 directly.

Two of the outputs 3 of the first therapeutic advice item 10 reference subsequent therapeutic advice items 11. The remaining output 3 of the first therapeutic advice item 10 references the next subsequent further therapeutic advice item 11 instead of the order feature 5 using therapeutic information 20 in the form of the link between the output identifier 7 of the referencing therapeutic advice item 10 and the input identifier 8 of the referenced therapeutic advice item 11. When an output 12 which references a further therapeutic advice item 11 is activated, this referenced therapeutic advice item 11 is also applied. If the referenced therapeutic advice item 11 comprises three outputs which themselves reference further therapeutic advice items 1, the chain of therapeutic advice items 1 is continued when the relevant output 3 is activated as output 12. This is done until finally at a last therapeutic advice item 1 an output 3 is activated which references no further therapeutic advice item 1. At this point, the recommendations A for action and/or decisions which underlie the generated therapeutic advice item 1 are output, if appropriate.

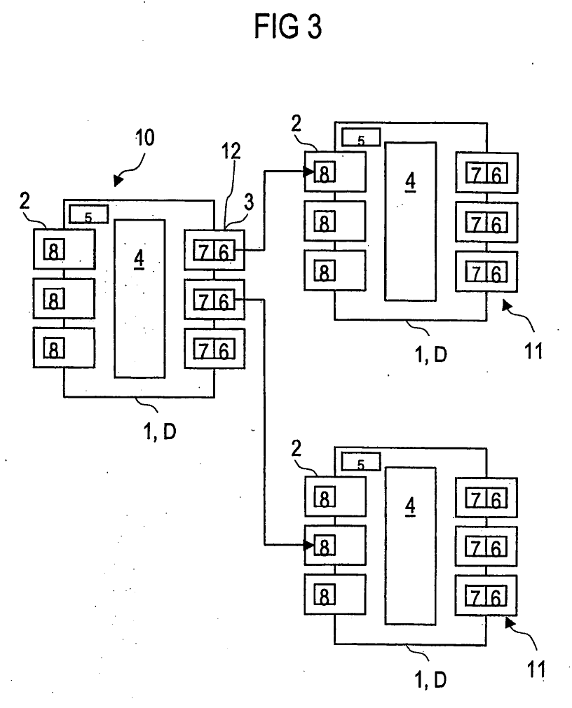

FIG. 3 shows an alternative form of referencing for therapeutic advice items 1. To be able to use this form of referencing, each output 3 or 12 of a therapeutic advice item 1 has an associated unique output identifier 7, and each input 2 of a therapeutic advice item 1 has an associated unique input identifier 8. To reference a subsequent therapeutic advice item 11, the input identifier 8 for an input 2 of the referenced therapeutic advice item 11 is entered for the output 12 of the referencing therapeutic advice item 10, that is to say as reference 6 in the form of the output identifier 7.

To create the references, provision is made for a user to be “offered”, e.g. shown in a selection menu, “suitable”, i.e. at least partially identical or similar (or identified as suitable in a database), order features 5 of further therapeutic advice items 11 as a function of the order feature 5 of the referencing therapeutic advice item 10. Selection of a therapeutic advice item 1 offered in this manner engages the reference 6 of the referencing therapeutic advice item 10 accordingly.

Alternatively, it is possible to “offer”, e.g. to show in a selection menu, “suitable”, that is to say partially identical or similar (or identified as suitable in a database), input identifiers 8 of further therapeutic advice items 11 for each output 3 or for each activated output 12 using the respective output identifier 7. Selection of a therapeutic advice item 1 offered in this manner engages the reference 6 of the referencing therapeutic advice item 10 accordingly.

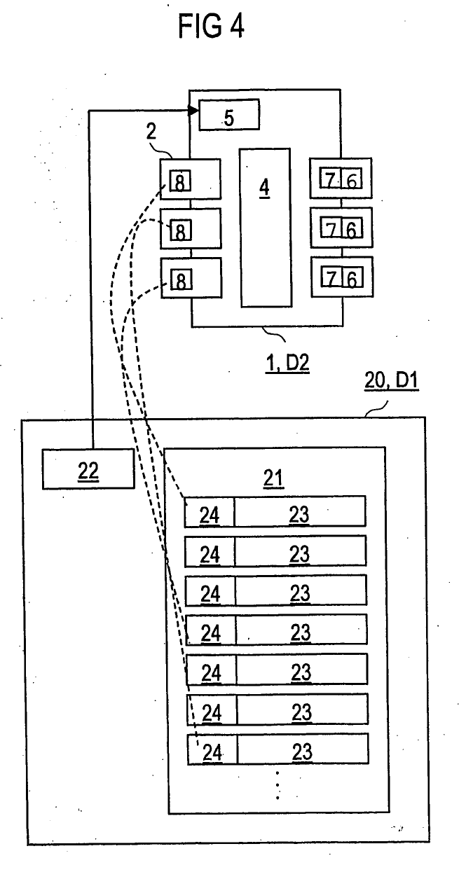

FIG. 4 illustrates that the input identifier 8 can also be used to select the respectively required data 23 for generating a therapeutic advice item 1 for a second data record D2 from a first data record D1 for therapeutic information 20, for example using an electronic patient record with all the relevant patient data 21 which forms the basis for a treatment. To this end, each data item 23 within the patient data 21, for example, has an associated unique identifier—data identifier 24.

Using the data identifier 24 and using the input identifier 8 of the therapeutic advice item 1, it is possible to retrieve the respectively required data 23 from the patient data 21 of the therapeutic information 20 as required. Alternatively or additionally, the relevant therapeutic information 20 of the first data record D1 may also be used for generating the therapeutic advice item of the second data record D2 with the aid of referencing using a reference 22. Depending on the type and design, the reference 22 includes at least portions of order features 5 of the referenced therapeutic advice item 1.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for linking data records including medical therapeutic advice items, comprising:
associating a respective data record associated with a medical therapeutic advice item including least one output; and
assigning an output a reference to a data record, for a subsequent medical therapeutic advice item in a treatment process.

2. The method as claimed in claim 1, wherein the respective data record for a therapeutic advice item is assigned an order feature provided for identifying the latter, and wherein the reference relates to the order feature of the data record for the therapeutic advice item to be referenced.

3. The method as claimed in claim 1, wherein the respective data record for a therapeutic advice item includes at least one input having an input identifier, and wherein the reference relates to an input identifier for the data record for the therapeutic advice item to be referenced.

4. The method as claimed in claim 1, wherein the data record for a therapeutic advice item includes a plurality of outputs, and wherein at least one output is activated.

5. The method as claimed in claim 2, wherein the data record for a therapeutic advice item is referenced by using the order feature of the data record for the referencing therapeutic advice item in order to select, automatically, data records for referenceable therapeutic advice items having partially identical order features and by proposing them to a user for selection, and wherein the data record for the referencing therapeutic advice item is assigned to the order feature of the therapeutic advice item selected in this manner.

6. The method as claimed in claim 3, wherein the data record for a therapeutic advice item is referenced by using an output identifier for the activated output of the data record for the referencing therapeutic advice item to select, automatically, data records for referenceable therapeutic advice items having an input identifier at least partially identical to the output identifier of the activated output and by proposing them to a user for selection, and wherein-the data record for the referencing therapeutic advice item is assigned, at its activated output, the input identifier of the therapeutic advice item selected in this manner.

7. A method for linking data records including medical therapeutic advice items, wherein a patient is treated by applying a first data record including therapeutic information and by prescribing at least one reference to a second data record for a therapeutic advice item,
wherein the therapeutic information includes at least patient data, the method comprising:
processing, for a referenced therapeutic advice item via the second data record selected patient data from the first data record;
using, for the referenced therapeutic advice item via the second data record, the patient data to activate at least one associated output; and
continuing, if the activated output includes a reference to a further second data record for another therapeutic advice item, the method with the processing step.

8. The method as claimed in claim 7, wherein the therapeutic information is stored in a determined format.

9. The method as claimed in claim 7, wherein the patient data are assigned an identifier.

10. The method as claimed in claim 8, wherein the data record for the therapeutic advice item includes at least one input and a format associated with this input or an identifier associated with this input, and wherein the data record for the therapeutic advice item in question uses the format or uses the identifier to select the patient data for processing.

11. The method as claimed in claim 7, wherein the therapeutic information includes patient data, and is stored in a prescribed format.

12. The method as claimed in claim 11, wherein the patient data are assigned an identifier.

13. The method as claimed in claim 9, wherein the data record for the therapeutic advice item includes at least one input and a format associated with this input or an identifier associated with this input, and wherein the data record for the therapeutic advice item in question uses the format or uses the identifier to select the patient data for processing.

14. The method as claimed in claim 12, wherein the data record for the therapeutic advice item includes at least one input and a format associated with this input or an identifier associated with this input, and wherein the data record for the therapeutic advice item in question uses the format or uses the identifier to select the patient data for processing.

15. A method for linking data records including medical therapeutic advice items, wherein a patient is treated by applying a first data record including therapeutic information and by prescribing at least one reference to a second data record for a therapeutic advice item as claimed in claim 1, wherein the therapeutic information includes at least patient data, the method comprising:
processing, for a referenced therapeutic advice item via the second data record, selected patient data from the first data record;
using, for the referenced therapeutic advice item via the second data record, the patient data to activate at least one associated output; and
continuing, if the activated output includes a reference to a further second data record for another therapeutic advice item, the method with the processing step.

16. The method as claimed in claim 15, wherein the therapeutic information is stored in a determined format.

17. The method as claimed in claim 15, wherein the patient data are assigned an identifier.
